(19) United States
(12) Patent Application Publication
(10) Pub. No.: US 2010/0028896 A1
(43) Pub. Date: Feb. 4, 2010

(54) ROTARY EXTRACTION CONTAINER AND METHOD OF IDENTIFYING CELL SPECIES, METHOD OF DETECTING GENE, AND AUTOMATIC NUCLEIC ACID EXTRACTOR USING THE SAME

(75) Inventors: Nao Noro, Tokyo (JP); Koji Miyazaki, Tokyo (JP)

Correspondence Address: CANTOR COLBURN, LLP, 20 Church Street, 22nd Floor, Hartford, CT, 06103, US

(73) Assignee: KONICA MINOLTA MEDICAL & GRAPHIC, INC., Tokyo (JP)

(21) Appl. No.: 12/519,448

(22) PCT Filed: Oct. 24, 2007
(86) PCT No.: PCT/JP2007/070717

§ 371 Date: Jun. 16, 2009

(30) Foreign Application Priority Data
Dec. 19, 2006 (JP) 2006-340913
Mar. 13, 2007 (JP) 2007-063226

(51) Int. Cl. C12Q 1/68; C12M 1/00; C12M 1/33

(52) U.S. Cl. 435/6; 435/283.1; 435/306.1

(57) ABSTRACT

Disclosed is a rotary extraction container enabling to safely and simply perform extraction and separation of a target substance from a sample containing plural substances. Specifically, there is disclosed a rotary extraction container enabling to simply perform extraction and separation of a nucleic acid from a biological sample or from a bio-derived sample without any risk of infection, contamination or the like, which has conventionally required cumbersome operations and a large, expensive apparatus. Further, there is disclosed a method of identifying a cell species a method of detecting a gene and an automatic nucleic acid extractor using the same. The foregoing rotary extraction container, which is a rotary extraction container to extract a target substance from a sample comprises a cylindrical container section, a rotating section and a cover section, and a solution or solid contained in any one of the small chambers of the cylindrical container section is allowed to transfer to another of the small chambers by an operation including rotation of the rotating section and the target substance is extracted from the sample by such an operation including the transfer.

### TECHNICAL FIELD

The present invention relates to a rotary extraction container to extract and separate a cell or nucleic acid from a biological sample or from a bio-derived sample and a method of identifying a cell species, a method of detecting a gene, and an automatic nucleic acid extractor using the same.

### TECHNICAL BACKGROUND

In general, when a sample containing plural substances is analyzed, it is frequently necessary to carry out an operation to extract and separate a specific object to be analyzed prior to analysis. For example, it is commonly necessary that a bio-derived sample such as blood or urine is subjected to an operation for extraction and separation as a pretreatment of a specimen prior to analysis, in order to remove unwanted components (e.g., proteins, lipids, and ionic substances) contained in the sample.

Since samples especially derived from clinical practice necessarily involve infection or contamination risk from viruses and bacteria, there has been desirable development of a method or a device to safely and rapidly conduct the entire pretreatment or even a partial pretreatment of such samples.

Accordingly, there have been proposed various types of extraction and separation methods. For example, to analyze a nucleic acid contained in a biochemical sample, there have been proposed extraction and separation methods utilizing a container of a special structure or magnetic particles (as set forth in, for example, Patent Documents 1-4).

However, these methods require cumbersome operations for a pretreatment of samples, and, in addition, are unable to overcome various problems such as infection or contamination risk produced treatment of samples derived from the above-described clinical practice.

On the other hand, there have been developed, over recent years, systems wherein devices and means to carry out conventional sample preparation, chemical analysis, and chemical synthesis (e.g., a pump, a valve, a flow channel, and a sensor) are miniaturized and integrated on a single chip by employing micromachine technology and microfabrication technology. These are referred to as μ-TAS's (Micro Total Analysis Systems), microchips, bioreactors, lab-on-chips, or biochips, which are expected to be applied in the fields of medical examination/diagnosis, environmental measurement, and agricultural production.

Especially, as shown in genetic testing, when cumbersome steps, skillful manipulation, and instrumental operations are required, it is assumed that an automatic, high-speed, and simple micro-analysis system is very beneficial, since analysis can be realized, without depending on time and location, as well as cost, the necessary sample quantity, and required duration.

However, in the above micro-analysis system, the greatest challenge required for a microchip to conduct testing thereon is that trace amount analysis is realized only with a minimal needed amount of a sample and a small amount of a reagent. However, some samples have a dilute concentration of a gene or nucleic acid, as a detection object. Since the amount of a specimen introducible into a chip is also limited, such a specimen amount does not fall within the measurable range. Accordingly, prior to introduction into the chip, a preliminary concentrating or separating operation is required. Optionally, it is necessary to mount, on a chip, a mechanism to detect or quantify a slight amount of a reaction product at high sensitivity via a simple operation. In detection of a gene, amplification reaction via a PCR (polymerase chain reaction) is commonly utilized. When a biological liquid such as blood is used as a sample, such a biological fluid does not often serve directly as a specimen for analysis, and in general, a certain pretreatment is frequently required.

For instance, there are employed chemical or physical method to extract or separate a nucleic acid from a biological sample. As methods relating to the latter, there were disclosed a method of extracting a nucleic acid from a cell via the action of vibrating beads (as set forth in, for example, Patent Document 5); and a method of separating and concentrating via application of an electrical field (as set forth in, for example, Patent Documents 6 and 7). Various problems, however, arose with application of such methods directly to microchips as an ultrafine device.

Therefore, also in the analysis field employing a microchip realizing such a simple and rapid testing means, specific problems and demands to be solved with respect to pretreatment such as extraction and separation are raised, and are being expected to be solved.

Patent Document 1: Japanese Translation of PCT International Application Publication JP 2001-511644W

Patent Document 2: JP 10-508100W

Patent Document 3: JP 2003-516156W

Patent Document 4: JP 2003-516156W

Patent Document 5: JP 2003-522521W

Patent Document 6: Japanese Patent Application Publication JP 2004-217A

Patent Document 7: WO 02/23180 Pamphlet

### DISCLOSURE OF THE INVENTION

### Problems to be Solved by the Invention

In view of the above problems and demands, the present invention has come into being. It is an object of the present invention to provide a rotary extraction container enabling to safely and simply perform extraction and separation of a target substance from a sample containing plural substances. Specifically, it is an object of the invention to provide a rotary extraction container enabling to simply perform extraction and separation of a nucleic acid from a biological sample or from a bio-derived sample without any risk of infection, contamination or the like, which has conventionally required cumbersome operations and a large, expensive apparatus. Further, it is an object of the invention to provide a method of identifying a cell species, a method of detecting a gene, and an automatic nucleic acid extractor using the same.

### Means to Solve the Problems

The above problems of the present invention were solved by the following means:

1. A rotary extraction container to extract a target substance from a sample,
(i) comprising a cylindrical container section, a rotating section and a cover section,
(ii) wherein the cylindrical container section has at least two small chambers,
(iii) the rotating section is closely attachable to the cylindrical container section and has an opening portion to connect any one of the small chambers with an outside,
(iv) the cover section is capable of sealing the opening portion of the rotating section, and
(v) a solution or solid contained in any one of the small chambers of the cylindrical container section is allowed to transfer to another of the small chambers by an operation including rotation of the rotating section and the target substance is extracted from the sample by such an operation including the transfer.

2. The rotary extraction container, as described in item 1, wherein the operation including a transfer allows the solid or a solid onto which is adsorbed the target substance to be collected.

3. The rotary extraction container as described in item 1 or 2, wherein the solid is a solid support holding the target substance or a material containing the target substance, the solid support is a solid support exhibiting magnetism (a magnetic support) and the magnetic support or a magnetic support onto which adsorbs the target substance is collected by applying a magnet to any portion of the rotary extraction container.

4. The rotary extraction container as described in any one of items 1-3, wherein the target substance is a nucleic acid and a material containing the target substance is a cell.

5. The rotary extraction container as described in item 4, wherein the nucleic acid is a nucleic acid of a microorganism belonging to chlamydias (Chlamydia), gonococci (Neisseria), or mycobacteria (Mycobacterium).

6. The rotary extraction container as described in any one of items 1-5, wherein the sample is a biological sample or a bio-derived sample.

7. The rotary extraction container as described in item 6, wherein the bio-derived sample is urine, blood, a cell suspension, or a sputum.

8. The rotary extraction container as described in any one of items 1-7, wherein a nucleic acid allowed to be eluted from a cell existing in the rotary extraction container by heating a part of or a whole of the rotary extraction container.

9. The rotary extraction container as described in any one of items 1-7, wherein a nucleic acid is allowed to be eluted from a cell existing in the rotary extraction container by applying ultrasonic to a part of or a whole of the rotary extraction container.

10. The rotary extraction container as described in any one of items 1-9, wherein at least one of magnetic particles, a washing solution, and a suspending solution is previously encapsulated in any one of the small chambers to extract the target substance from the sample.

11. The rotary extraction container described in any of items 1-10 wherein a harvesting step, a washing step, and a lysis step are conducted in a plurality of small chambers of the cylindrical container section whereby a nucleic acid is extracted.

12. The rotary extraction container as described in any one of items 1-11, wherein a dripping orifice is provided in any portion of the rotary extraction container and the target substance extracted from the sample is allowed to drip from a dripping orifice.

13. A method of identifying a cell species, wherein a nucleic acid extracted and obtained using the rotary extraction container described in any one of items 1-12 is identified by a nucleic acid amplification method.

14. A detecting method of a gene comprising amplifying a nucleic acid extracted by a rotary extraction container described in any of items 1-12 in a device having a microchip to detect the gene.

15. An automatic nucleic acid extractor, wherein a nucleic acid is automatically extracted by a rotary extraction container described in any of items 1-12.

### EFFECTS OF THE INVENTION

A rotary extraction container which can safely and simply extract and separate a target substance from a sample containing a plurality of substances can be provided by the foregoing means of the present invention. Especially, there can be provided a rotary extraction container enabling to simply extract and separate, without contamination or biohazard risk, a nucleic acid from a biological sample (or a bio-derived sample), which has conventionally required cumbersome operations and a large, expensive apparatus, a method of identifying a cell species, a method of detecting a gene, and an automatic nucleic acid extractor using the same.

### BRIEF DESCRIPTION OF THE DRAWINGS

FIG. 1 is a schematic view showing a constitution of the rotary extraction container of the present invention

FIG. 2 is a schematic view showing an example of a nucleic acid extraction method

FIG. 3 is a schematic view showing an example of a dripping method of an extract liquid

FIG. 4 is a schematic view showing an example of a microchip for nucleic acid amplification detection

### DESCRIPTION OF THE ALPHANUMERIC DEGIGNATIONS

A: cylindrical container section

A1, A2, and A3: small chambers

B: rotating section

C: cover section

D: opening portion

1: micropump connection section

2: liquid supply control section

3: ultrafine flow channel

4: reagent containing section

5: specimen liquid (liquid containing an isolated nucleic acid)

6: specimen liquid accepting section

7: reagent

### PREFERRED EMBODIMENT OF THE INVENTION

The rotary extraction container of the present invention is a rotary extraction container to extract a target substance from a sample

(i) comprising a cylindrical container section, a rotating section and a cover section,

(ii) wherein the cylindrical container section has at least two small chambers,

(iii) the rotating section is closely attachable to the cylindrical container section and has an opening portion to connect any one of the small chambers with an outside,

(iv) the cover section can sealing the opening portion of the rotating section, and

(v) an operation including rotation of the rotating section allows a solution or solid contained in any one of the small chambers of the cylindrical container section to be transferred to another of the small chambers and such an operation including the transfer allows the target substance to be extracted from the sample.

These features are technological ones common to the invention relating to the foregoing item 1 to item 15.

Herein, the operation including rotation essentially requires a rotating operation and also refers to a rotating operation which optionally contains other operations as a series of operations such as an up-and-down shaking operation and a reversely rotating operation of the top and the bottom portions. Further, the operation including a transfer essentially requires a transfer operation and also refers to a transfer operation optionally containing other operations as a series of operations such as an operation to collect, via a magnetic force, a solid support having magnetism (a magnetic support) adsorbed with a target substance as described later.

Herein, a “solid” referred to in the present invention refers to a solid substance as a sample containing a target substance to be extracted; a solid substance as an objective substance to be extracted; or a solid support allowed to hold a target substance to be extracted or a target substance contained substance via adsorption (including chemical adsorption and physical adsorption).

The present invention and components thereof will be described in detail.

### Constitution of Rotary Extraction Container

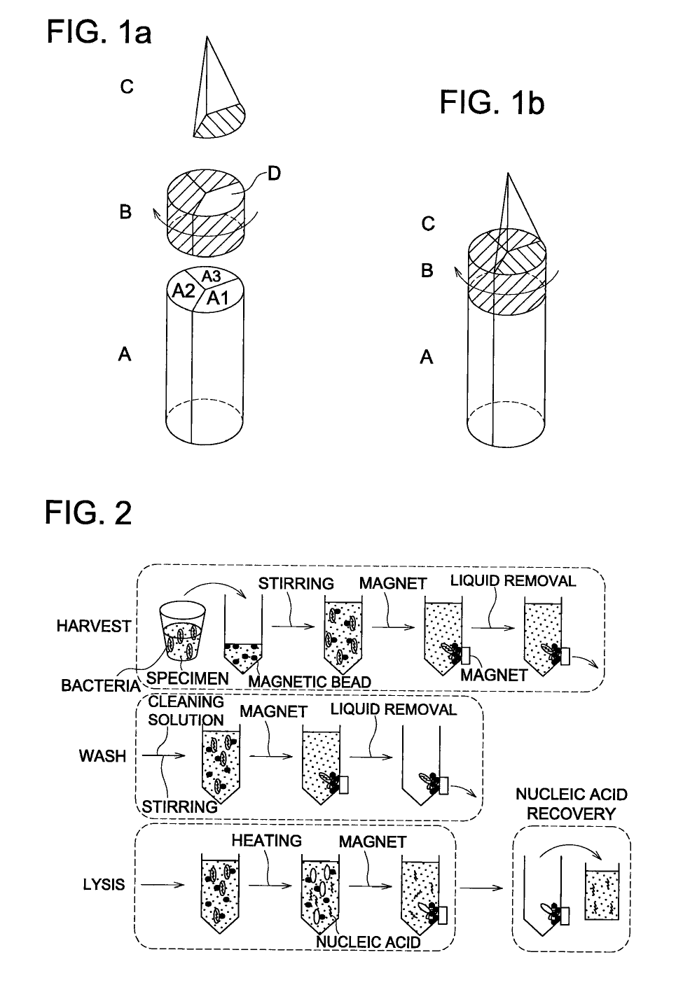

Description will now be made with reference to a schematic view of the rotary extraction container of the present invention, illustrated in FIG. 1. The rotary extraction container is basically constituted of a cylindrical container section A, a rotating section B, and a cover section C.

The cylindrical container section A has at least two small chambers (there are, for example, in FIG. 1, three small chambers including a small chamber A1 to a small chamber A3). Another small chamber may also be optionally provided depending on the purpose of use. As shown in an embodiment to be described later, there can be provided, for example, a small chamber for harvest, a small chamber for washing, or a small chamber for suspending.

The rotating section B is closely attachable to the cylindrical container section and has a portion to partially cover the small chambers and opening portion D (an opening area equal to that of the inlet of each small chamber) to connect any of the small chambers to the exterior. A solution or a suspension as a sample and a solid substance or magnetic particles as a solid support can be put in and taken out from any one of the small chambers of the cylindrical container section through an opening portion D.

A cover section C can simultaneously seal the cylindrical container section A by sealing the opening portion D of the rotating section B. Further, the cover section C also serves as an acceptor for a substance coming out from the opening portion D when the rotary extraction container is turned upside down.

The rotating section B of the rotary extraction container is rotated until the opening portion D comes directly above a small chamber containing a solution or solid, and thereafter the rotary extraction container is turned upside down to transfer the solution or solid from the small chamber to a cover section C through the opening portion D. Subsequently, the rotating section B is rotated so that the opening portion D comes directly above another small chamber, and then the solution or solid can be transferred from the cover section C to the another small chamber through the opening portion D. Therefore, a target substance can be extracted from a sample by operations including a rotational operation and a transfer operation.

Thus, in cases when the solid is a solid substance as a sample containing a target substance to be extracted, a solid substance as a target substance to be extracted or a solid support onto which is adsorbed a target substance to be extracted, the solid or the solid onto which is adsorbed a target substance can be collected by the foregoing operation including transfer.

Herein, when a solid is a solid support onto which is adsorbed a target substance or a material containing a target substance and the solid support is also a support exhibiting magnetism (hereinafter referred to as a “magnetic support”), the magnetic support or the magnet support onto which is adsorbed such a target substance or a material containing a target substance can be collected by applying a magnet to any portion of the rotary extraction container.

As materials to form the cylindrical container A, the rotating section B, and the cover section C according to the present invention, conventionally known materials such as metal or plastics are usable depending on the sample contents. Preferable materials include, for example, polypropylene, polyethylene, and polycarbonate.

Further, the size of the cylindrical container section A, the rotating section B, and the cover section C can be determined to be an appropriate one, depending on the sample contents, the sample amount, and the analysis apparatus.

### Operational Procedures of Rotary Extraction Container

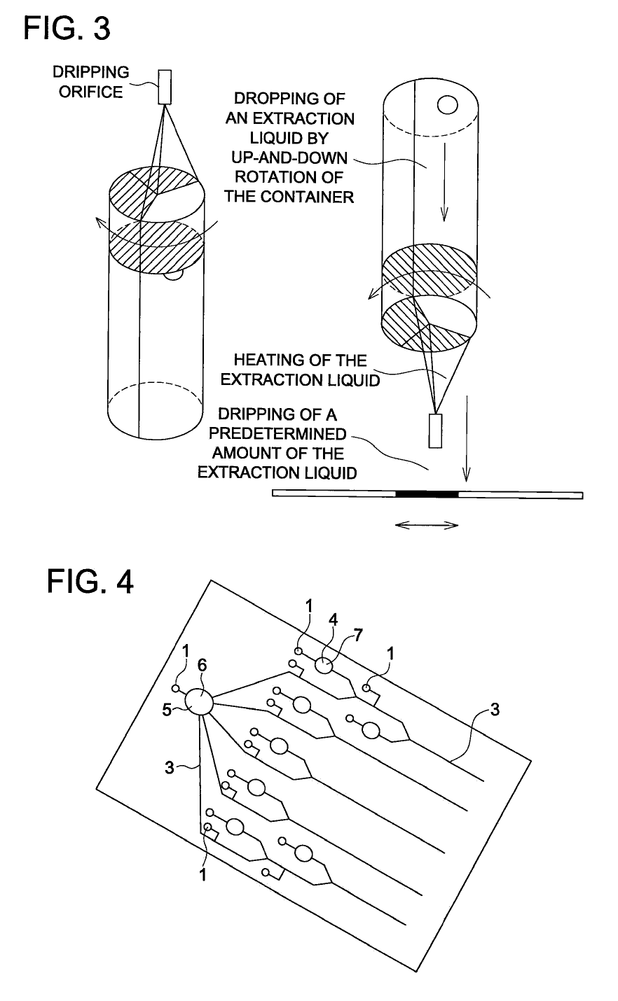

Operational procedures for use of the rotary extraction container of the present invention will now be described with reference to a typical example of the embodiments of the present invention (as shown in FIG. 2 and FIG. 3).

(1) A harvest solution (200 μl) and magnetic beads (30 μl at a concentration of 1 mg/ml) as a magnetic support are placed in a small chamber (A1) of the cylindrical container section A and a washing solution (1 ml) is placed in another small chamber (A2) in advance. Also, a lysis solution (100 μl) is previously placed in a small chamber (A3).

(2) A sample (1 ml of urine) is placed in the small chamber (A1) from the opening portion D of rotating section B and then cover section C is set for sealing.

(3) The sample (1 ml of urine), the harvest solution (200 μl), and the magnetic beads (30 μl) are mixed with stirring, followed by being left for 1 minute. Thus, bacteria are allowed to be adsorbed onto the magnetic beads.

(4) The rotary extraction container is turned upside down and the magnetic beads are collected and recovered (harvested) by applying a magnet to the cover section (30 seconds).

(5) The top and bottom portions of the rotary extraction container are returned. The rotating section B is rotated so that the opening portion D meets another small chamber (A2) and then the magnet is removed to transfer the magnetic beads to the small chamber (A2).

(6) In the small chamber (A2), 1 ml of the washing solution and the magnetic beads are mixed with stirring to perform washing.

(7) The rotary extraction container is turned upside down. The magnet is applied to cover section C (30 seconds) and the thus cleaned magnetic beads are recovered.

(8) The top and bottom portions of the rotary extraction container are returned. Rotating section B is rotated so that the opening portion D meets another small chamber (A3) and then the magnet is removed to transfer the magnetic beads to the small chamber (A3).

(9) In the small chamber (A3), the lysis solution (100 μl) and the magnetic beads are mixed with stirring.

(10) The rotary extraction container is turned upside down. Cover section C is placed in a heater and heated under a predetermined condition for temperature and duration (at 94° C. for 1 minute) to dissolve the bacteria (or to extract a nucleic acid).

(11) While the magnetic beads are held by applying the magnet to cover section C (30 seconds), the top and bottom portions of the rotary extraction container are returned and the solution is returned to the small chamber (A3).

(12) The cover section C is removed and the nucleic acid extraction liquid is recovered using a micropipette, dropper or the like. Alternatively, a dripping orifice is provided in the cover section C and then the nucleic acid extraction liquid is dripped onto a microchip for nucleic acid amplification detection from the dripping orifice. Such a dripping orifice can be provided in any portion of the rotary extraction container such as the small chamber (A3) but is preferably provided in cover section C. Dripping from cover section C can achieve elimination of an instrument such as a pipette or dropper and instrumental procedures, reduction of operational mistakes, and simplification of operations. For example, testing of multiple times or for multiple items can be carried out only with a given amount (e.g., 25 μl) of a lysis solution (100 μl). The dripping portion is preferably shaped similarly to an eye drop container. A predetermined amount of a solution can be taken out from a container safely and accurately via a simple operation, provided that the solution can be pushed out as droplets similarly to an eye drop.

Incidentally, the foregoing small chambers (A1), (A2), and (A3) correspond to a small chamber for harvest, a small chamber for washing, and a small chamber for suspending, respectively. In order to separate the above target substance from a sample as described in the foregoing example, at least one of magnetic beads, a washing solution, and a suspending liquid is preferably placed in any one of the small chambers in advance, from the viewpoint of safety and simplicity.

Further, rotating section B is designed to rotate in the order of (A1), (A2), and (A3) and not to rotate adversely. In addition, rotating section B is provided with a stopper to prevent removal thereof. Also, cover section C is preferably provided with a stopper so as not to be removed once set.

As is obvious from the above embodiment, while a single rotary extraction container is sealed, treatment and operations of a harvest step, a washing step, and a lysis step are carried out sequentially in each of the small chambers ((A1)-(A3)) of the cylindrical container section A and the cover section C.

In the present invention, a “harvest step” refers to allowing cell, as a target substance, to adsorb onto a solid support from a harvest solution. A “harvest solution” refers to a solution prepared by previously dissolving cell in a solvent so that the cell is adsorbed onto a solid support, and herein, a cell suspension is considered to be included therein. A “washing step” refers to a step of washing to remove an excess solvent and reagent from a solid support adsorbed with cell. Further, a “lysis step” refers to a step in which cell adsorbed to a solid support are heated and then cell walls or cell membranes are destroyed to elute a nucleic acid into a solvent. A “lysis solution” refers to a solution to elute a nucleic acid by destroying cell walls or cell membranes.

When a nucleic acid is extracted using the rotary extraction container of the present invention, various reagents and a magnetic support (magnetic beads) are needed as described above. These reagents also include a dissolving liquid or a diluting liquid to dissolve or dilute a sample, a washing solution, and various types of buffer solutions.

Extraction and isolation of a nucleic acid require various types of buffer solutions. For example, as binding buffer solutions (e.g., a harvest solution), there are exemplified buffer solutions composed of salts such as ammonium acetate, sodium chloride, potassium chloride, sodium acetate, or potassium acetate and alcohol such as methanol, ethanol, isopropanol, or n-butanol. Further, as washing buffer solutions (washing solutions), those prepared via 4- to 5-fold dilution of any of the above binding buffer solutions may be used. Alternatively, another buffer solution of different type may be prepared separately. Water is preferable as a suspending liquid.

In one of the preferred embodiments of the present invention, it is desirable to previously enclose a set of instruments and materials needed such as the above magnetic support and various types of reagents into the rotary extraction container as a kit.

As can be seen from the foregoing examplified embodiments, using the rotary extraction container of the present invention, a nucleic acid can simply be extracted and separated, without contamination or biohazard risk, from a biological sample or from a bio-derived sample, which has conventionally required cumbersome operations and a large, expensive apparatus.

### Automatic Nucleic Acid Extractor

The rotary extraction container of the present invention can be simply operated and therefore is usable as a device carrying out a series of operations automatically, and is specifically suitable for an automatic nucleic acid extractor automatically extracting a nucleic acid. Thereby, a nucleic acid can further simply be extracted and separated without contamination or biohazard risk.

Further, this automatic nucleic acid extractor which is built in a nucleic acid analyzer can also perform a series of operations needed to analyze a nucleic acid, automatically from beginning to end.

### Sample and Target Substance

When an a target substance (also referred to as an “extraction object”) is extracted and separated from a sample using the rotary extraction container of the present invention, such a sample and a target substance are not limited to any specific substances and a wide variety of substances can be used. Especially, the present invention can remarkably come into effect, when the following biological sample or bio-derived sample is used as a sample and then a cell and a nucleic acid contained therein are target substances to be extracted and separated.

In the present invention, a target substance to be extracted, namely, cells to be targets to be extracted, include any one of cells or cell cultures of microorganisms (e.g., bacteria, fungi, and yeasts), plants, and animals without specific limitation. Microbial cells are preferable and cells of microorganisms belonging to chlamydia (Chlamydia), gonococcus (Neisseria), or mycobacterium (Mycobacterium) are desirable.

Any sample, if being a sample containing the above cell and also a bio-derived sample, is not specifically limited, including most bio-derived samples such as whole blood, plasma, serum, buffy coat, urine. fecal matter, saliva, sputum, cerebral spinal fluid, semen, tissue (e.g., cancerous tissue and lymph node), and cell culture fluid (e.g., mammal cell culture and bacterial culture). There are targetted nucleic acid-containing samples, 'samples possibly incorporating or containing microorganisms, and all other samples possibly containing nucleic acids (e.g., foods and biological formulations). Further, there are also cited environmental samples possibly containing living organisms such as soil or drainage water. The form of such a sample is preferably a fluid sample and is usually a liquid such as a solution or a suspension. The sample may be a soluble solid or a solid floating in a liquid.

In the present invention, nucleic acids as a target to be extracted exist in the form of deoxyribonucleic acid (DNA) and ribonucleic acid (RNA). DNA includes, for example, plasmid DNA, complementary DNA (cDNA), and genomic DNA. RNA includes, for example, messenger RNA (mRNA), transfer RNA (tRNA), and ribosomal RNA (rRNA). Herein, a single strand or a double strand does not matter. The amount of DNA to be isolated is preferably in the range of 0.001-1 mg.

In the present invention, a “gene” refers to a nucleic acid carrying genetic information exerting any kind of function, that is, DNA or RNA, and also to the form of DNA or RNA expressed as only a chemical substance. Further, a “base” refers to the nucleic acid base of nucleotide.

Various physical methods known in the art can be used for the above destruction of cell membranes. Cell destruction is preferably carried out by heating. The reason is that heating is simple and as described above, it is unnecessary to later remove a chemical agent been used for cell membrane destruction. Specifically, the foregoing heating is conducted in the temperature range in which no nucleic acid is denatured by heating, namely, from 70 to 120° C., preferably from 80 to 120° C., more preferably from 80 to 100° C. over a period of from 20 seconds to 10 minutes, preferably from 20 seconds to 300 seconds. Heating conditions (temperature and time) vary depending of the kind of a cell or bacteria (size, composition and thickness of the cell membrane), and therefore are appropriately selected within the above range. Heating is carried out via any appropriate heating method. Examples thereof include a dry heat block, a hot water bath, a microwave oven, and various types of heaters but are not limited to these heating methods.

In addition to the steps described above, there may further be included a step to concentrate a nucleic acid isolated through evaporation of water by heating. Heating is conducted within the temperature range in which the nucleic acid is not denatured. The foregoing cell membrane destruction is conducted by heating so that the cell membrane destruction step by heating can also serve as a concentration step.

As is obvious from the above, in suitably applicable embodiments of the rotary extraction container of the present invention, a target substance to be extracted is specifically preferably a cell or a nucleic acid from the viewpoint of the object of the present invention and the problems to be solved thereby, as well as the effects of the present invention. Therefore, as a sample is specifically preferred a biological sample (or a bio-derived sample) as described above. Further, as such a biological sample (or a bio-derived sample) is specifically preferred urine, blood, cell suspension, or sputum.

### Solid Support

As described above, the “solid” defined in the present invention includes a solid substance as a sample containing a target substance to be extracted, a solid material as a target substance to be extracted and a solid support which hold a target substance to be extracted through adsorption. Of these solids, the solid support relating to the present invention is preferably a water-insoluble carrier. Such a carrier is preferably one which exhibits magnetism (hereinafter also referred to as a “magnetic support”).

In the present invention, water-insoluble materials to form a water-insoluble solid support are not specifically limited and any water-insoluble material is usable. Water insolubility referred to herein specifically refers to a solid phase insoluble in water or an aqueous solution containing any water-soluble composition. The solid support may be any one of supports or matrix known in the art which have been now widely used for immobilization or separation, or proposed so far.

Specific examples include an inorganic compound, a metal, a metal oxide, an organic compound, and a composite material prepared by the combination of the foregoing. A target substance such as a cell contained in a sample is adsorbed onto a solid support, but such a solid support is not specifically limited with respect to material, shape and size of the solid support are not specifically limited, provided that the target substance such as a cell can be adsorbed thereto. Preferred examples include a material providing a large surface area for cell binding, namely for nucleic acid binding.

Specifically, materials used for a solid support are not specifically limited, but in general include a synthesized organic polymer such as polystyrene, polypropylene, polyacrylate, polymethylmethacrylate, polyethylene, polyamide, or latex; an inorganic substance such as glass, silica, silicon dioxide, silicon nitride, zirconium oxide, aluminum oxide, sodium oxide, calcium oxide, magnesium oxide, or zinc oxide; and metal such as stainless steel or zirconia. These materials usually have irregular surfaces, e.g., porous or granular, and therefore can be used, including, for example, particles, a fiber, a web, a sintered material, or a sieved material.

Accordingly, the shape of a solid support used in the present invention is not specifically limited, including a granular shape, a rod shape, a plate shape, a sheet, a gel, a film, a fiber, a capillary, a strip, and a filter. Of these, a granular shape is preferable. A granular material, for example, beads are generally preferable in terms of large binding force.

The granular shape includes, for example, a spherical form, an elliptical form, a conical form, a cubic form, and a rectangular parallelepiped form. Of these, a spherical particle carrier is of preferred in terms of being easily produced and rotation-stirring of a magnetic support being easily performed when used. The average particle diameter of beads serving as a magnetic support onto which is adsorbed with a target substance such as a cell, is from 0.5 to 10 μm, preferably from 2 to 6 μm. In the case of an average particle diameter of less than 0.5 μm, when the bead body is formed by incorporating a magnetic material, sufficient magnetic responsibility has not come into effect, a substantially long period of time is required to separate the particles, and a substantially large magnetic force is also required for the separation. In contrast, in the case of a particle diameter of more than 10 μm, the particles are easily sedimented in an aqueous medium so that an operation to stir the medium is required during cell capturing. Further, the surface area of the particle body becomes small, often rendering it difficult to capture cells in a sufficient amount.

The entire bead including the surface may be constituted of a single material and also may be a hybrid body constituted of plural materials as need. For example, to respond to analysis automation, there are exemplified composite beads in which the core portion is made of a magnetically responding material such as ferric oxide or chromium oxide and the surface thereof is covered with a synthesized organic polymer.

From the viewpoint that a magnetic support bonded with a cell is easily allowed be subjected to (solid-liquid) separation and particle recovery from a sample liquid by the magnetic force of a magnet, such a magnetic support preferably contains a magnetic material such as a paramagnetic material, para-ferromagnetic material or ferromagnetic material, and more preferably contains both or at least one of a paramagnetic material and a para-ferromagnetic material. Of these, a para-ferromagnetic material is specifically preferred in terms of no residual magnetization or a small amount thereof.

Specific examples of such a magnetic material include ferrosoferric oxide (Fe₃O₄), γ-ferric oxide (γ-Fe₂O₃), various types of ferrites, metal such as iron, manganese, cobalt, or chromium, and various types of alloys of cobalt, nickel, and manganese. Of these, ferrosoferric oxide is specifically preferable.

It is preferable that a magnetic support used in the present invention be in the form of beads made of particles of small particle diameter and exhibit excellent magnetic separation properties (namely, performance to be separated via magnetism over a short period of time), as well as being easily suspended via a gentle up-and-down shaking operation.

The content of a magnetic material in the magnetic beads is usually not more than 70% by mass, preferably from 20 to 70% by mass, more preferably from 30 to 70% by mass, since the content ratio of a non-magnetic organic substance is at least 30% by mass. A content of less than 20% by mass results in insufficient sufficient magnetic responsiveness, frequently rendering it difficult to separate particles in a short period of time via a required magnetic force. On the other hand, when this ratio exceeds 70% by mass, the amount of a magnetic material exposed on the particle body surface increases, leading to elution of some components of the magnetic material such as iron ions. Thereby, other materials may adversely be affected during use and no practical strength may often be achieved since the particle body becomes fragile.

In the extraction method of the present invention, a sample liquid containing a cell and a magnetic support (preferably magnetic beads) are mixed and the cell is adsorbed (including chemical adsorption and physical adsorption) onto the magnetic support, whereby the cell can efficiently be accumulated on the surface of the support. Even when the cell is not adsorbed onto the magnetic support, the cell can be accumulated via magnetic or centrifugal force. Desirably, the cell is adsorbed onto the magnetic support, but may not be adsorbed thereto.

Some cells, specifically bacterial cells are not adsorbed to a magnetic support. To further assuredly accelerate adsorption or adhesion of a cell, it is possible to attach, to the surface of a magnetic support, a group exhibiting affinity to the cell, a reactive functional group such as an amino group, an oxycarbonylimidazole group, an N-hydroxysuccinic acid imide group, or a “functional substance” such as sugar, a sugar protein, an antibody, lectin or a cell adhesion factor specifically exhibiting affinity to a target cell. There may be performed appropriate coating to accelerate modification of the surface structure of the magnetic support or binding.

In a sample in which the concentration of a cell contained therein, specifically a target bacterial cell is small, a large amount of a sample liquid is treated, nevessitating operations such as separation and concentration are required. According to the method of the present invention in which a cell is allowed to bond or adhere to a magnetic support and a nucleic acid in the cell is easily extracted, such sample treatment can rapidly be carried out through simple operations. In the present invention, solid-liquid separation utilizing magnetic beads and a magnet together with a detachable cover is extremely convenient specifically in the case of a small amount of a sample. In such a case, due to the loss of a cell or a nucleic acid in the course of separation and extraction, the final yield of the target nucleic acid may fall below an amount applicable to analysis. However, in the method of the present invention, such a loss during isolation is hardly generated. In the method of the present invention, there is not used any chemical agent such as a chaotrope reagent, a surfactant, or a solvent bacterium which influences nucleic acid amplification reaction, hybridization, restriction enzyme reaction, detection reaction, or electrophoresis analysis, so that a separated (isolated) nucleic acid as such can be subjected to amplification reaction. Therefore, according to the method of the present invention, even with a trace amount of a sample, a nucleic acid can be separated (isolated) from a cell with high yield and high purity.

### Nucleic Acid Amplification

The rotary extraction container of the present invention can suitably be used as a method of identifying a cell species in which an extracted and isolated nucleic acid is amplified through a nucleic acid amplification method to identify the nucleic acid. Thus, using the rotary extraction container of the present invention for the above identification method, an extraction and separation (isolation) operations essential for the method can be carried out easily, rapidly, and safely.

Specifically, a nucleic acid extracted and isolated from a bacterial cell contained in a sample is amplified through a DNA amplification method such as PCR (Polymerase Chain Reaction), SDA (Strand Displacement Amplification), LCR (Ligase Chain Reaction), ICAN (Isothermal and Chimeric Primer-Initiated Amplification of Nucleic Acids), LAMP (Loop-Mediared Isothermal Amplification), TMA (Transcription-Mediated Amplification), TAS (Transcription Amplification System), or 3SR (Self-Sustained Sequence Replication System), NASBA (Nucleic Acid Sequence-Based Amplification). The thus amplified nucleic acid is analyzed, for example, via a base sequence determination method, a hybridization method, or a Southern blotting method and then the type of the bacterial cell can be identified by comparison with the standard or target base sequence.

### Genetic Testing Method

The rotary extraction container of the present invention is suitably applicable to a gene testing method incorporating steps to amplify and detect a nucleic acid (gene) in a device having a microchip. Namely, using the rotary extraction container of the present invention for the above gene testing method, an extraction and separation (isolation) operation essential for the method can be carried out easily, rapidly, and safely.

A nucleic acid analysis device to conduct the gene testing method of the present invention may include a microchip-shaped one, whereby high throughput analysis can be carried out.

### Nucleic Acid Analysis Device

A nucleic acid analysis device to conduct the gene testing method of the present invention is composed of a device body in which a micropump, a controller to control the micropump, and a temperature controller to control temperature are united and a microchip for nucleic acid amplification detection attachable to this device body. A specimen liquid is injected into the specimen acceptor of the microchip in which a reagent has been previously encapsulated. The microchip is mounted on the nucleic acid analysis device body and then mechanical connection to activate a liquid sending pump is made, if appropriate, along with electrical connection for controlling. A microchip flow channel is activated via the connection between the body and the microchip. Accordingly, in one example of the preferred embodiments, once an operation is initiated, supplying and mixing of a specimen and a reagent, nucleic acid amplification and detection are automatically carried out through a series of continuous steps.

A unit serving as a control system to control each of liquid supplying, mixing, and temperature, together with a micropump, constitutes the nucleic acid analysis device body of the present invention. This device body is commonly used for a specimen by mounting the above microchip thereon. The above steps such as liquid mixing, liquid supplying, and nucleic acid amplification and detection are built in software, programmed along with controlling of the micropump and temperature, which is mounted on the nucleic acid analysis device as preset conditions for liquid sending order, volume, and timing. In the present invention, it is only necessary to replace the microchip which is detachable. The nucleic acid analysis device of the present invention features downsizing of every component and a shape able to be conveniently carried, whereby no place or time for use is limited and then excellent workability and operability are realized. Since many micropump units for use in liquid supplying are built in the device body, the microchip can be used as a disposable type.

### Microchip for Nucleic Acid Amplification Detection, Micropump, and Pump Connection Section

As one example of the preferred embodiments of a microchip for nucleic acid amplification detection, the embodiment shown in FIG. 4 will now be described. A specimen accepting section 6 and a reagent containing section 4 are provided with micropumps to supply the liquid contents of these containing sections. Each micropump is connected to the upstream side of the regent containing section 4 via a pump connection section 1, and a driving liquid is fed toward the reagent containing section side by the micropump, whereby a reagent is pushed out into a flow channel for liquid supplying. Such microchip pump units are built in the nucleic acid analysis device body, independent of the microchip for nucleic acid amplification detection. By mounting the microchip on the nucleic acid analysis device body, pump connection section 1 is connected to the microchip.

In one of the embodiments of the present invention, a piezo pump is used as a micropump. Thus, such a piezo pump is one provided with a first flow channel in which flow channel resistance varies with the differential pressure, a second flow channel in which the rate of flow channel resistance variation due to differential pressure variation is smaller than that of the first flow channel, a pressurizing chamber connected to the first flow channel and the second flow channel, and an actuator to vary the inner pressure of the pressurizing chamber. The detail is described in Japanese Patent Application Publication JP 2001-322099A and JP 2004-108285A.

There will now be described one example of the preferred embodiments of a chip for nucleic acid amplification detection used for the afore-described nucleic acid analysis device. A microchip of the embodiment is one in which there are provided at least a specimen liquid accepting section 6, a reagent containing section 4, a waste liquid reservoir, a micropump connection section 1, and a ultrafine flow channel 3; these sections each are communicated with one another via ultrafine flow channels; specimen liquid 5(liquid containing an isolated nucleic acid) is allowed to flow through a flow channel constituting a nucleic acid amplification section provided in the downstream of the specimen accepting section and then through a flow channel constituting a section to detect an amplified nucleic acid; the nucleic acid is analyzed by mixing with a reagent 7 contained in the reagent containing section 4; and a resulting waste liquid is transferred to and confined in the waste liquid reservoir. Further, in addition to each of the containing sections, the flow channels, and the pump connection sections, each element such as a liquid sending section, a backward flow prevention section, a reagent quantifying section, and a mixing section is functionally provided in appropriate locations by microfabrication technology.

Next, one example of the preferred embodiments of a microchip will now be illustrated. A microchip for nucleic acid amplification detection is a microchip sheet produced by appropriate combination of at least one member selected from a plastic resin, glass, silicon, and ceramics. The horizontal and vertical sizes thereof are usually about several 10 mm and several mm in height. Ultrafine flow channels and the frame body of the microchip are formed with a plastic resin, which is easily processed and formed, as well as being inexpensive and easy in incineration disposal. Specifically, a resin such as polyolefin, e.g., polypropylene, or polystyrene is desirable due to excellent moldability. The ultrafine flow channels are formed with a size of approximately from 10 to several 100 μm in width and height, for example, with a width of approximately 100 μm and a depth of approximately 100 μm.

### Nucleic Acid Amplification and Detection

A nucleic acid isolated using the rotary extraction container of the present invention is amplified by the nucleic acid amplification section of a microchip for nucleic acid amplification detection and then the thus amplified nucleic acid is transferred to the detection section of the microchip to detect the nucleic acid (gene). Nucleic acid amplification is carried out through a DNA amplification method such as PCR, SDA, LCR, ICAN, LAMP, TMA, TAS, 3SR, or NASBA, as described earlier. The amplified nucleic acid is analyzed via a common method such as a hybridization method or a colloidal gold adsorption method.

The entire part or a part of the microchip and the nucleic acid analysis device can be modified to any type of variation, provided that the structure, constitution, arrangement, shape, size, material, system, and method thereof meet the object of the present invention.

Incidentally, the rotary extraction container of the present invention is built in the nucleic acid analysis device as the automatic nucleic acid extractor as described above. Thereby, a series of operations required for nucleic acid analysis can be performed automatically from beginning to end and further easily with no contamination and biohazard risk.

As mentioned above, the present invention has been described with reference to the drawings shown as examples of typical embodiments of the present invention. The present invention is not limited to such embodiments and examples.

1. A rotary extraction container to extract a target substance from a sample,
(i) comprising a cylindrical container section, a rotating section and a cover section,
(ii) wherein the cylindrical container section has at least two small chambers,
(iii) the rotating section is closely attachable to the cylindrical container section and has an opening portion to connect any one of the small chambers with an outside,
(iv) the cover section is capable of sealing the opening portion of the rotating section, and
(v) a solution or solid contained in any one of the small chambers of the cylindrical container section is allowed to transfer to another of the small chambers by an operation including rotation of the rotating section and the target substance is extracted from the sample by such an operation including the transfer.

2. The rotary extraction container as claimed in claim 1, wherein the operation including a transfer allows the solid or a solid onto which is adsorbed the target substance to be collected.

3. The rotary extraction container as claimed in claim 1, wherein the solid is a solid support holding the target substance or a material containing the target substance, the solid support is a solid support exhibiting magnetism (a magnetic support) and applying a magnet to any portion of the rotary extraction container allows the magnetic support or the magnetic support onto which is adsorbed the target substance to be collected.

4. The rotary extraction container as claimed in claim 1, wherein the target substance is a nucleic acid and the material containing the target substance is a cell.

5. The rotary extraction container as claimed in claim 4, wherein the nucleic acid is a nucleic acid of a microorganism from the group consisting of chlamydias (Chlamydia), gonococci (Neisseria) and mycobacteria (Mycobacterium).

6. The rotary extraction container as claimed in claim 1, wherein the sample is a biological sample or a bio-derived sample.

7. The rotary extraction container as claimed in claim 6, wherein the bio-derived sample is a urine, a blood, a cell suspension, or a sputum.

8. The rotary extraction container as claimed in claim 1, wherein heating a partial portion of or an entire portion of the rotary extraction container allows a nucleic acid to be eluted from a cell existing in the rotary extraction container.

9. The rotary extraction container as claimed in claim 1, wherein applying ultrasonic to a part of or a whole of the rotary extraction container allows a nucleic acid to be eluted from a cell existing in the rotary extraction container.

10. The rotary extraction container as claimed in claim 1, wherein at least one of magnetic particles, a washing solution, and a suspending solution is previously-encapsulated in any one of the small chambers to extract the target substance from the sample.

11. The rotary extraction container as claimed in claim 1 wherein a harvesting step, a washing step, and a lysis step are conducted in a plurality of small chambers of the cylindrical container section to extract a nucleic acid.

12. The rotary extraction container as claimed in claim 1 wherein a dripping orifice is provided in any portion of the rotary extraction container and the target substance extracted from the sample is allowed to drip from a dripping orifice.

13. A method of identifying cell species, wherein a nucleic acid which has been extracted and obtained using the rotary extraction container as claimed in claim 1 is identified by a nucleic acid amplification method.

14. A detecting method of a gene comprising amplifying a nucleic acid extracted by a rotary extraction container as claimed in claim 1 in a device having a microchip to detect the gene.

15. An automatic nucleic acid extractor, wherein a nucleic acid is automatically extracted by a rotary extraction container as claimed in claim 1.
